(19) United States
(12) Patent Application Publication
(10) Pub. No.: US 2024/0335365 A1
(43) Pub. Date: Oct. 10, 2024

(54) COMPOSITION COMPRISING GLYCEROL FATTY ACID ESTERS AND FATTY ACID AMIDOALKYL BETAINES AND/OR ALKYL BETAINES

(71) Applicant: Evonik Operations GmbH, Essen (DE)

(72) Inventors: Dominik Schuch, Duesseldorf (DE); Marlene Hübner, Muelheim an der Ruhr (DE); Peter Schwab, Essen (DE); Joachim Venzmer, Essen (DE); Jochen Kleinen, Heinsberg (DE)

(73) Assignee: Evonik Operations GmbH, Essen (DE)

(21) Appl. No.: 18/580,010

(22) PCT Filed: Jul. 11, 2022
(86) PCT No.: PCT/EP2022/069237

§ 371 Date: Jan. 17, 2024

(30) Foreign Application Priority Data
Jul. 21, 2021 (EP) 21186918.5

(51) Int. Cl. A61K 8/44; A61K 8/37; A61Q 19/10

(52) U.S. Cl. CPC A61K 8/442; A61K 8/375; A61Q 19/10; A61K 2800/48

(57) ABSTRACT

Compositions that have glycerol fatty acid esters and particular fatty acid amidoalkyl betaines and/or particular alkyl betaines can be used for the thickening of cosmetic formulations. The composition can also be used for thickening an aqueous formulation. The composition also has 3.0% by weight to 20% by weight of glycerol fatty acid esters, wherein the weight percentages are based on the overall composition.

### FIELD OF THE INVENTION

The invention provides compositions comprising glycerol fatty acid esters and particular fatty acid amidoalkyl betaines and/or particular alkyl betaines, and also the use of these compositions for the thickening of cosmetic formulations.

### PRIOR ART

A large number of cosmetic surfactant formulations offered on the market contain fatty acid amidopropyl betaines having a coconut oil-derived alkyl chain distribution (INCI: Cocamidopropyl Betaine) as a foam-providing component in combination with a thickener that may for example be a glycerol fatty acid ester.

The combination of cocamidopropyl betaine and glyceryl monolaurate has established itself on the market as a very effective combination and aqueous premixtures have accordingly been sold for many years by Evonik under the tradenames ANTIL HS 60 and TEGO Betain HS KB 5. Since glyceryl monolaurate is a solid, the advantage of the liquid premixtures lies in their easy processability into the end formulations.

However, both products, which represent the current prior art, each have a disadvantage.

The highly concentrated aqueous mixture ANTIL HS 60, which comprises about 20% by weight of glyceryl monolaurate, 28% by weight of cocamidopropyl betaine, 5% by weight of sodium chloride and 2% by weight of glycerol, is indeed a good thickener, but the turbid mixture has a yield point at temperatures between 10 and 40° C. As a result, after preparation and filling into containers, air bubbles are stabilized and there may be agglomeration and flotation of extremely small amounts (ppb) of activated carbon—introduced from the refining of the triglyceride—which are then visible as black particles on the product surface and possibly also in the end formulation.

TEGO Betain HS KB 5 is a less-concentrated aqueous solution composed of about 5% by weight of glyceryl monolaurate, 24% by weight of cocamidopropyl betaine, 4.5% by weight of sodium chloride, 2% by weight of glycerol and 0.5% by weight of sodium benzoate, which does not have a yield point at temperatures between 10 and 40° C. such that no flotation and particle formation occurs here. However, compared to ANTIL HS 60, the product has a much lower, and for commercial surfactant formulations insufficient, thickening performance.

US20080261842 discloses cleansing compositions in the form of a gel for use in an aerosol container, the composition comprising:
(a) a base material which consists at least of a surfactant in an amount not less than 7% by weight of the total composition and a thickener that is a blend of at least one glyceryl ester and a glyceryl ester derivative with at least one of a betaine and a gum, said base material having a viscosity greater than 9,500 cps; and
(b) a foam forming material, above 9% by weight of said composition, at least a part of the foam forming material being maintained in suspension in the composition until after the composition is dispensed from the aerosol, wherein the foam forming material is a saturated aliphatic hydrocarbon having from 4 to 5 carbons, and wherein the composition is in the form of a gel prior to inclusion of the foam forming material.

An object of the present invention was to provide a composition comprising betaine and glycerol fatty acid ester, which does not have a yield point between 10 and 40° C. and at the same time functions as a good thickener for cosmetic surfactant systems.

Surprisingly, it has been found that both of the above-described disadvantages from the prior art can be overcome, such that a product with a thickening performance comparable to ANTIL HS 60—with respect to the product concentration—is obtained as a clear solution without yield point between 10 and 40° C.

Relevant for the achievement of the object of the invention is inter alia the composition of the betaine component, which in particular is based on tropical oils such as coconut oil and palm kernel oil.

Coconut oil differs from palm kernel oil in its fatty acid chain distribution, in particular in the oleic acid content, which is generally from 5% by weight to 10% by weight for native coconut oil and generally from 10% by weight to 20% by weight for palm kernel oil, based on all fatty acids present in the triglyceride. In addition, the caprylic and capric acid content in palm kernel oil is lower than that in coconut oil, as can be seen from Tables A and B.

Hydrogenated coconut fat is generally used for the preparation of commercial betaines with the result that the majority of the acyl radicals having 18 carbon atoms are present in the form of stearic acid. The hydrogenated coconut oils that are used for the preparation of the betaines and are present in the above-mentioned products thus have an oleic acid acyl radical content of less than 5% by weight.

TABLE A
Fatty acid chain distribution (figures in percent by weight) of coconut fat
and palm kernel fat (both not hydrogenated) based on gas chromatography
analyses according to Codex Alimentarius of the Food and Agriculture
Organization of the United Nations (CODEX STAN 210-1999)

| | Fatty acid radical | | | | | | | |
|---|---|---|---|---|---|---|---|---|
| | C8:0 | C10:0 | C12:0 | C14:0 | C16:0 | C18:0 | C18:1 | C18:2 |
| Coconut oil | 4.6-10.0 | 5.0-8.0 | 45.1-53.2 | 16.8-21.0 | 7.5-10.2 | 2.0-4.0 | 5.0-10.0 | 1.0-2.5 |
| Palm kernel oil | 2.4-6.4 | 2.6-5.0 | 45.0-55.0 | 14.0-18.0 | 6.5-10.0 | 1.0-3.0 | 12.0-19.0 | 1.0-3.5 |

TABLE B
Fatty acid chain distribution (figures in percent by weight) of coconut fat
and palm kernel fat (both not hydrogenated) based on gas chromatography analyses
according to Thieme RÖMPP Online, Georg Thieme Verlag, 2014

| | Fatty acid radical | | | | | | | |
|---|---|---|---|---|---|---|---|---|
| | C8:0 | C10:0 | C12:0 | C14:0 | C16:0 | C18:0 | C18:1 | C18:2 |
| Coconut oil | 5-10 | 5-8 | 45-53 | 17-21 | 7-10 | 2-4 | 5-10 | <2 |
| Palm kernel oil | 3 | 5 | 47-52 | 16 | 6-9 | 2-3 | 10-18 | 1-3 |

### DESCRIPTION OF THE INVENTION

It has surprisingly been found that the compositions described hereinbelow are able to achieve the object of the invention and are able to overcome at least one of the disadvantages of the prior art.

The present invention therefore provides compositions comprising glycerol fatty acid esters and particular fatty acid amidoalkyl betaines and/or particular alkyl betaines according to claim 1.

The invention further provides for the use of the compositions according to the invention for the thickening of cosmetic formulations.

One advantage of the present invention is the effective thickening performance, in particular in surfactant formulations.

One advantage of the present invention is the very good processability, in particular in cosmetic surfactant formulations.

A further advantage is the absence of a yield point between 10 and 40° C.

A further advantage is the lack of formation of solid particles.

A further advantage is the clarity and homogeneity of the mixture, as a result of which separation effects can be avoided.

A further advantage is the ratio of betaine to glycerol ester in the premixture, this already being suitable for commercial surfactant formulations, which simplifies the production of end formulations.

A further advantage is the mildness of the surfactant mixture, as a result of which the mildness of a primary surfactant-containing end formulation can be improved by synergistic effects.

A further advantage is the positive influence on the skin feel and the foam properties of an end formulation, for example as a result of an improved creaminess of the foam due to a reduction of the average air bubble diameter in the foamed formulation.

The present invention provides compositions comprising
A) at least one glycerol fatty acid ester and
B) at least one betaine component selected from
B1) fatty acid amidoalkyl betaine of the general formula I)

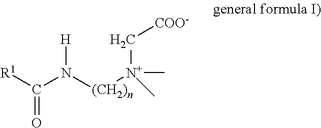

where
n=1 to 10, preferably 2 to 5, in particular 3, and
R¹CO=mixture of acyl radicals having 6 to 30, in particular 8 to 22, carbon atoms, characterized in that the mixture of acyl radicals has a total content of oleic acid acyl radicals and linoleic acid acyl radicals, based on all acyl radicals of the mixture, of 5% by weight to 50% by weight, preferably of 12% by weight to 25% by weight, particularly preferably of 13% by weight to 20% by weight, and
B2) alkyl betaine of the general formula II)

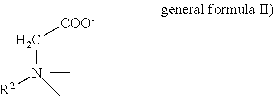

where
R²=saturated or unsaturated, optionally hydroxy-substituted alkyl radical having 6 to 22 carbon atoms, or mixtures thereof,
characterized in that the weight ratio of all glycerol fatty acid esters present in the composition to all fatty acid amidoalkyl betaines and alkyl betaines present in the composition is from 1.0:1.5 to 1.0:8.0, wherein component B) is selected in particular from B1).

The contents of glycerol fatty acid esters and glycerol can be determined by means of the GC-FID method described hereinbelow after derivatization with N-methyl-N-(trimethylsilyl)trifluoroacetamide.

Unless otherwise indicated, all stated percentages (%) are percentages by mass.

The proportion by mass of glycerol and of the glycerol partial esters can be determined within the context of the present invention by a GC method; this method involves derivatization of the composition according to the invention to the greatest possible extent and then simultaneous determination by means of GC/FID.

To this end, 0.10 g of the product according to the invention together with in each case 3 mg of propanediol and 20 mg of 1-pentadecanol as internal standards are dissolved in 5 ml of pyridine:chloroform (4:1). 0.25 ml of this solution is admixed with 0.5 ml of MSTFA [N-methyl-N-(trimethylsilyl)trifluoroacetamide]. The alcohols are quantitatively converted to their trimethylsilyl ethers by reaction at 80° C. (30 minutes) and then analysed by means of GC/FID.

This is performed in a gas chromatograph equipped with a split/splitless injector, a capillary column and a flame ionization detector, under the following conditions:
Injector: 290° C., split 40 ml
Injected volume: 1 μl
Column: 30 m*0.32 mm DB5-HT 0.1 μm
Carrier gas: hydrogen, const. flow, 2 ml/min
Temperature program: 65° C. at 10° C./min to 365° C.
   then conditioning at 365° C. for 15 minutes.
Detector: FID at 365° C.
   Hydrogen 35 ml/min
   Air 240 ml/min
   Make-up gas 12 ml/min

Glycerol, glycerol ester and propane-1,3-diol and 1-pentadecanol as internal standards are separated.

By evaluating the peak area of the glycerol compared to the peak area of the propane-1,3-diol added as internal standard, and by evaluating the peak area of the glycerol partial ester compared to the peak area of the 1-pentadecanol added as internal standard, the proportions by mass of glycerol and glycerol partial ester can be determined.

For this, the GC system is calibrated by analysing mixtures of the glycerol or glycerol partial ester to be investigated and of the internal standards with known composition.

The betaine contents can be determined in accordance with the article entitled “Titrimetric methods for the determination of betains” published in Application Bulletin.—Metrohm AG, No. 264/1 d.

It is preferable according to the invention that in B1) R¹CO is a mixture of acyl radicals having 6 to 30 carbon atoms originating from a natural oil or fat; the radicals R¹CO are therefore in particular acyl radicals of natural fatty acids.

Fatty acids can be produced on the basis of natural such as for example vegetable or animal oils and have preferably 6-30 carbon atoms, especially 8-22 carbon atoms. Fatty acids are generally unbranched and usually have an even number of carbon atoms. Any double bonds have cis configuration. Examples are: caproic acid, caprylic acid, capric acid, lauric acid, myristic acid, palmitic acid, palmitoleic acid, isostearic acid, stearic acid, 12-hydroxystearic acid, dihydroxystearic acid, oleic acid, linoleic acid, linolenic acid, petroselinic acid, elaidic acid, arachic acid, behenic acid, erucic acid, gadoleic acid, linolenic acid, eicosapentaenoic acid, docosahexaenoic acid, arachidonic acid.

In the present case, component B1) is a mixture of fatty acid amidoalkyl betaines.

The respective acyl radical content of component B1) can be determined experimentally for example by means of a combination of two liquid chromatography methods. In this case, the individual betaines of the mixture, which contain various numbers of carbon atoms in the acyl radical, are first separated by means of HPLC/RI according to a method described by R. Gerhards et al. (Modern methods for the analysis of cocamidopropyl betaines; Gerhards. R et. al.; Tenside, Surfactants, Detergents; 1996 33(1):1-12).
Since this method gives an insufficient peak separation for the betaines having 16 to 18 carbon atoms (C16, C18:0, C18:1 and C18:2) in the acyl radical, if oleylamidopropyl betaine components are present, this component is separated by means of a second gradient HPLC method. This is carried out on an RP column (reversed phase column, Hypersil Gold 100 mm*3 mm; 5μ, Thermofisher) at 50° C. in a gradient of 0.05% aqueous ammonium formate solution (pH adjusted to 4.2 with formic acid) and acetonitrile (gradient programme: start with 10% acetonitrile maintained for 1 minute; then a 15 minute linear gradient from 10% to 95% acetonitrile; then maintained for 10 minutes at 95% acetonitrile), the individual betaine fractions being detected by CAD (CAD=charged aerosol detector, Dionex Corona Ultra RS, Thermo Scientific).

The acyl radical contents of the individual betaines the acyl radicals of which contain 8 to 14 carbon atoms, and also the sum of the acyl radical contents of the betaines the acyl radicals of which contain 16 to 18 (C16, C18:0, C18:1 and C18:2) carbon atoms, can consequently be determined from the peak area percentages of the HPLC/RI measurement.

The acyl radical contents of the individual betaines the acyl radicals of which contain 16 to 18 (C16, C18:0, C18:1 and C18:2) carbon atoms then result from the peak area percentages thereof from the HPLC/CAD measurement after standardizing to the sum of the peak area percentages of these four betaines from the HPLC/RI measurement. For this, the peak area percentage value of the respective betaine from the HPLC/CAD measurement is multiplied by the sum of the peak area percentages of these four betaines from the HPLC/RI measurement and then divided by the sum of the peak area percentages of these four betaines from the HPLC/CAD measurement.

The alkyl radical contents of component B2) can be determined in an analogous fashion.

The terms “coconut fat” and “coconut oil” are used synonymously.

The terms “palm kernel fat” and “palm kernel oil” are used synonymously.

Unless otherwise indicated, all stated percentages (%) are percentages by mass.

Compositions preferred according to the invention are characterized in that they comprise 3.0% by weight to 20% by weight, preferably 5.5% by weight to 15% by weight, particularly preferably 6.5% by weight to 10% by weight, of glycerol fatty acid esters, where the weight percentages are based on the overall composition.

It is preferable according to the invention that the composition of the present invention comprises in total 10% by weight to 35% by weight, preferably 12% by weight to 31% by weight, particularly preferably 14% by weight to 27% by weight, of fatty acid amidoalkyl betaines and alkyl betaines, where the weight percentages are based on the overall composition.

In this context it is particularly preferable that the composition of the present invention comprises in total 10% by weight to 35% by weight, preferably 12% by weight to 31% by weight, particularly preferably 14% by weight to 27% by weight, of fatty acid amidoalkyl betaines of the general formula I) and alkyl betaines of the general formula II), where the weight percentages are based on the overall composition.

Compositions preferred according to the invention are characterized in that the glycerol fatty acid ester, based on all acyl radicals present in the glycerol fatty acid ester, includes at least 90% by weight acyl radicals having 8 to 18 carbon atoms. It is particularly preferable according to the invention that, based on all acyl radicals having 8 to 18 carbon atoms present in the glycerol fatty acid ester, at least 50% by weight of them are lauroyl radicals.

Compositions particularly preferred according to the invention are characterized in that the glycerol fatty acid ester, based on all glycerol fatty acid ester, contains at least 70% by weight glycerol fatty acid monoesters.

It is preferable according to the invention that the mixture of acyl radicals in the fatty acid amidoalkyl betaine of the general formula I) has a content of acyl radicals having 8 to 18 carbon atoms of at least 90% by weight, where the weight percentages are based on all acyl radicals in the mixture.

It is preferable according to the invention that the mixture of acyl radicals in the fatty acid amidoalkyl betaine of the general formula I) contains the amounts of the respective acyl radical listed below, given in weight percentages, where further acyl radicals not listed here may be present and the weight percentages mentioned are based on all acyl radicals present in the mixture:

| C8:0 | C10:0 | C12:0 | C14:0 | C16:0 | C18:0 | C18:1 | C18:2 |
|---|---|---|---|---|---|---|---|
| 0-10 | 0-10 | 30-99 | 0-30 | 0-20 | 0-10 | 10-30 | 0-20 |

It is preferable according to the invention that the mixture of acyl radicals in the fatty acid amidoalkyl betaine of the general formula I) has a content of oleic acid acyl radicals, based on all acyl radicals of the mixture, of 10% by weight to 30% by weight, preferably of 12% by weight to 21% by weight, particularly preferably of 13% by weight to 17% by weight.

It is preferable according to the invention that the mixture of acyl radicals in the fatty acid amidoalkyl betaine of the general formula I) has a content of linoleic acid acyl radicals (C18:2), based on all acyl radicals of the mixture, of 0.5% by weight to 5.0% by weight, preferably of 1.0% by weight to 4.0% by weight.

A component B1) particularly preferred according to the invention is characterized in that the mixture of acyl radicals in the fatty acid amidoalkyl betaine present has a content of oleic acid acyl radicals, based on all acyl radicals of the mixture, of 12% by weight to 21% by weight, and a content of linoleic acid acyl radicals, based on all acyl radicals of the mixture, of 1.5% by weight to 4.0% by weight.

It is very particularly preferable according to the invention that the mixture of acyl radicals in the fatty acid amidoalkyl betaine of the general formula I) corresponds to the acyl radical distribution of a palm kernel oil as can be seen from Tables A and B, where the respective lowest and highest weight percentage limit value per acyl radical from both tables applies.

Compositions particularly preferred according to the invention are characterized in that R² in the alkyl betaines of the general formula II) includes in total at least 50% by weight lauryl, myristyl and cetyl radicals, preferably at least 50% by weight lauryl radicals, where the weight percentages are based on all alkyl radicals in the alkyl betaines, especially of the general formula II), present in the composition.

It is preferable according to the invention that the composition of the present invention comprises 33% by weight to 83% by weight, particularly preferably 48% by weight to 78% by weight, of water, where the weight percentages are based on the overall composition.

The water content is determined by means of the Karl Fischer titration familiar to those skilled in the art in accordance with DIN 51777 and DGF C-III 13a.

Compositions preferred according to the invention are characterized in that they comprise in total 0.1% by weight to 10% by weight of sodium chloride and/or potassium chloride, where the weight percentages are based on the overall composition.

The salt content can be determined in accordance with DGF H-III 9.

It is preferable according to the invention that the composition of the present invention comprises 0.1% by weight to 25% by weight of glycerol, where the weight percentages are based on the overall composition.

Compositions preferred according to the invention are characterized in that they comprise in total 0.1% by weight to 3% by weight of fatty acids selected from fatty acids having 8 to 18 carbon atoms, where the weight percentages are based on the overall composition.

The fatty acid content can for example be determined by means of HPLC analysis according to M. J. Cooper, M. W. Anders. Anal. Chem., 1974, 46 (12), pp. 1849-1852.

It is preferable according to the invention that the composition of the present invention does not have a yield point between 10 and 40° C.

The existence of a yield point is determined as described below in the examples.

The composition of the present invention preferably has, according to the invention, a pH in a range from 3.1 to 12.9, preferably 3.6 to 7.9, particularly preferably 4.1 to 6.9.

The “pH” in connection with the present invention is defined as the value which is measured at 25° C. after stirring for 5 minutes using a pH electrode calibrated in accordance with ISO 4319 (1977).

In addition, impurities and by-products of technical-grade betaine qualities known to those skilled in the art and also preservatives may be present in the composition of the present invention in each case at up to 1% by weight, where the weight percentages are based on the overall composition, for example unconverted amine radicals, glycolic acid or sodium benzoate.

The invention further provides for the use of at least one composition of the present invention for the thickening of a formulation, in particular a cosmetic, preferably an aqueous, and very particularly preferably a surfactant-containing, aqueous, formulation.

The present invention further provides formulations, especially in the form of a cosmetic, pharmaceutical or dermatological formulation.

The formulations of the invention can further comprise at least one additional component selected from the group of
emollients,
emulsifiers,
surfactants,
thickeners/viscosity regulators/stabilizers,
UV light protection filters,
antioxidants,
hydrotropes (or polyols),
solids and fillers,
film formers,
pearlescence additives,
deodorant and antiperspirant active substances,
insect repellents,
self-tanning agents,
preservatives,
conditioning agents,
perfumes,
colourants,
odour absorbers,
cosmetic active substances,
care additives,
superfatting agents, and
solvents,
in particular at least one surfactant, preferably at least one surfactant and water.

Substances which can be used as exemplary representatives of the individual groups are known to those skilled in the art and can be taken, for example, from German patent application DE 102008001788.4. This patent application is hereby incorporated by reference and is accordingly considered to form part of the disclosure.

As regards further optional components and also the amounts of these components employed, reference is expressly made to the relevant handbooks known to those skilled in the art, for example K. Schrader, “Grundlagen und Rezepturen der Kosmetika” [Fundamentals and Formulations of Cosmetics], 2nd edition, pages 329 to 341, Hüthig Buch Verlag, Heidelberg. The amounts of the respective additives depend on the intended use.

Typical starting formulations for the relevant applications are known prior art and are contained for example in the brochures of the manufacturers of the relevant base materials and active substances. These existing formulations can generally be adopted unchanged. However, if necessary, for adjustment and optimization, the desired modifications can be made in a straightforward manner through simple tests.

The examples that follow describe the present invention by way of example, without any intention to limit the invention, the scope of application of which is apparent from the entirety of the description and the claims, to the embodiments specified in the examples.

### EXAMPLES

For the sake of better comparability of the thickening performance of the compositions prepared in accordance with the following examples, comparable active contents were used in the test formulations. The active content is used here analogously to the term “dry residue”, which can be determined by those skilled in the art. It results from 100% minus the water content of the respective sample determined by means of Karl Fischer titration in accordance with DIN 51777, DGF E-III 10 and DGF C-III 13 a. Alternatively, the dry residue can be determined in accordance with DGF B-II 3/C-III 12. For Example 2, in which glycerol was used as further solvent, the glycerol content added, in addition to the water content, was subtracted from 100% in order to determine the active content.

### Example 1 According to the Invention

A solution of 114.8 g of a fatty acid amidopropyl betaine based on refined palm kernel oil (containing 15.8% oleic acid acyl radicals and 2.2% linoleic acid acyl radicals based on all acyl radicals of the betaine, further composed of 55.3% water, 33.6% palm kernel fatty acid amidopropyl betaine with the INCI name Cocamidopropyl Betaine, 7.1% sodium chloride, 2.5% glycerol and 1.5% fatty acids from palm kernel oil) and 68.8 g of water was heated to 80° C. With stirring, 16.4 g of glyceryl monolaurate (here and in further examples: technical grade quality containing at least 90% glyceryl monolaurate) were added in portions within 30 min and then the mixture was stirred at 80° C. for a further 30 min. The mixture was then allowed to cool to 22° C. The product was a clear, pale yellowish, homogeneous liquid having a viscosity of 3500 mPa·s.

The active content was 33.9% with a ratio of the components A to B of 1 to 2.35.

### Example 2 According to the Invention

A solution of 70.8 g of C12/C14 alkyl betaine (composed of 62.4% water, 30.8% Coco-Betaine (INCI) and 6.8% sodium chloride) and 16.7 g of glycerol was heated to 80° C. With stirring, 12.5 g of glyceryl monolaurate were added in portions within 30 min and then the mixture was stirred at 80° C. for a further 30 min. The mixture was then allowed to cool to 22° C. The product was a clear, pale yellowish, homogeneous liquid having a viscosity of 950 mPa·s.

The active content was 43.3% with a ratio of the components A to B of 1 to 1.74.

### Example 3 According to the Invention

A solution of 81.0 g of C12/C14 alkyl betaine (composed of 62.4% water, 30.8% Coco-Betaine (INCI) and 6.8% sodium chloride) and 10.0 g of water was heated to 80° C. With stirring, 9.0 g of glyceryl monolaurate were added in portions within 30 min and then the mixture was stirred at 80° C. for a further 30 min. The mixture was then allowed to cool to 22° C. The product was a clear, pale yellowish, homogeneous liquid having a viscosity of 600 mPa·s.

The active content was 39.5% with a ratio of the components A to B of 1 to 2.77.

### Example 4 Not in Accordance With the Invention

96.0 g of a fatty acid amidopropyl betaine based on hydrogenated coconut oil (containing <1% unsaturated fatty acid acyl radicals based on all acyl radicals of the betaine, further composed of 55.0% water, 34.0% Cocamidopropyl Betaine (INCI), 6.5% sodium chloride, 2.5% glycerol and 2.0% fatty acids from coconut oil) were heated to 80° C. With stirring, 22.0 g of glyceryl monolaurate were added in portions within 30 min and then the mixture was stirred at 80° C. for a further 30 min. The mixture was then allowed to cool to 22° C. The product was a turbid, slightly yellowish liquid having a viscosity of 3400 mPa·s, which in contrast to the examples according to the invention stabilized air bubbles present in the product over a period of >24 hours. This may result in floatation and particles formation by activated carbon residues, which in particular on a relatively large scale become visible on the surface.

The active content was 55.5% with a ratio of the components A to B of 1 to 1.48.

### Example 5 Not in Accordance With the Invention

95.0 g of a fatty acid amidopropyl betaine based on hydrogenated coconut oil (containing <1% unsaturated fatty acid acyl radicals based on all acyl radicals of the betaine, further composed of 67.0% water, 26.0% Cocamidopropyl Betaine (INCI), 4.8% sodium chloride, 1.6% glycerol and 0.6% fatty acids from coconut oil) were heated to 80° C. With stirring, 5.0 g of glyceryl monolaurate were added in portions within 30 min and then the mixture was stirred at 80° C. for a further 30 min. The mixture was then allowed to cool to 22° C. The product was a clear, pale yellowish liquid having a viscosity of 80 mPa·s.

The active content was 36.4% with a ratio of the components A to B of 1 to 4.94.

### Example 6 Not in Accordance With the Invention

96.0 g of a fatty acid amidopropyl betaine based on refined palm kernel oil (containing 15.8% oleic acid acyl radicals and 2.2% linoleic acid acyl radicals based on all acyl radicals of the betaine, further composed of 55.3% water, 33.6% palm kernel fatty acid amidopropyl betaine with the INCI name Cocamidopropyl Betaine, 7.1% sodium chloride, 2.5% glycerol and 1.5% fatty acids from palm kernel oil) were heated to 80° C. With stirring, 23.0 g of glyceryl monolaurate were added in portions within 30 min and then the mixture was stirred at 80° C. for a further 30 min. The mixture was then allowed to cool to 22° C. The product was a turbid, slightly yellowish liquid having a viscosity of 6700 mPa·s, which in contrast to the examples according to the invention stabilized air bubbles present in the product over a period of >24 hours. Furthermore, the mixture showed a phase separation after 4 days at 22° C.

The active content was 55.4% with a ratio of the components A to B of 1 to 1.40.

### Example 7 Not in Accordance With the Invention

A solution of 96.0 g of a fatty acid amidopropyl betaine based on refined palm kernel oil (containing 15.8% oleic acid acyl radicals and 2.2% linoleic acid acyl radicals based on all acyl radicals of the betaine, further composed of 55.3% water, 33.6% palm kernel fatty acid amidopropyl betaine with the INCI name Cocamidopropyl Betaine, 7.1% sodium chloride, 2.5% glycerol and 1.5% fatty acids from palm kernel oil) and 72.0 g water was heated to 80° C. With stirring, 23.0 g of glyceryl monolaurate were added in portions within 30 min and then the mixture was stirred at 80° C. for a further 30 min. The mixture was then allowed to cool to 22° C. The product was a turbid, slightly yellowish liquid having a viscosity of 2200 mPa·s, which in contrast to the examples according to the invention stabilized air bubbles present in the product over a period of >24 hours. Furthermore, the mixture showed a phase separation after 48 hours at 22° C.

The active content was 34.5% with a ratio of the components A to B of 1 to 1.40.

### Example 8 Not in Accordance With the Invention

A solution of 96.5 g of a fatty acid amidopropyl betaine based on refined palm kernel oil (containing 15.8% oleic acid acyl radicals and 2.2% linoleic acid acyl radicals based on all acyl radicals of the betaine, further composed of 55.3% water, 33.6% palm kernel fatty acid amidopropyl betaine with the INCI name Cocamidopropyl Betaine, 7.1% sodium chloride, 2.5% glycerol and 1.5% fatty acids from palm kernel oil) and 30.0 g water was heated to 80° C. With stirring, 3.5 g of glyceryl monolaurate were added in portions within 30 min and then the mixture was stirred at 80° C. for a further 30 min. The mixture was then allowed to cool to 22° C. The product was a clear, pale yellowish liquid having a viscosity of 90 mPa·s.

The active content was 35.9% with a ratio of the components A to B of 1 to 9.26.

### Example 9 According to the Invention

A solution of 114.8 g of a fatty acid amidopropyl betaine based on refined coconut oil (containing 6.1% oleic acid acyl radicals and 1.5% linoleic acid acyl radicals based on all acyl radicals of the betaine, further composed of 54.2% water, 34.3% Cocamidopropyl Betaine (INCI), 6.8% sodium chloride, 2.6% glycerol and 2.1% fatty acids from coconut oil) and 68.8 g of water was heated to 80° C. With stirring, 16.4 g of glyceryl monolaurate (here and in further examples: technical grade quality containing at least 90% glyceryl monolaurate) were added in portions within 30 min and then the mixture was stirred at 80° C. for a further 30 min. The mixture was then allowed to cool to 22° C. The product was a clear, pale yellowish, homogeneous liquid having a viscosity of 3100 mPa·s.

The active content was 34.5% with a ratio of the components A to B of 1 to 2.40.

### Example 10 Not in Accordance With the Invention

A solution of 96.0 g of a fatty acid amidopropyl betaine based on refined coconut oil (containing 6.1% oleic acid acyl radicals and 1.5% linoleic acid acyl radicals based on all acyl radicals of the betaine, further composed of 54.2% water, 34.3% Cocamidopropyl Betaine (INCI), 6.8% sodium chloride, 2.6% glycerol and 2.1% fatty acids from coconut oil) and 81.0 g water was heated to 80° C. With stirring, 23.0 g of glyceryl monolaurate were added in portions within 30 min and then the mixture was stirred at 80° C. for a further 30 min. The mixture was then allowed to cool to 22° C. The product was a turbid, slightly yellowish liquid having a viscosity of 1750 mPa·s, which in contrast to the examples according to the invention stabilized air bubbles present in the product over a period of >24 hours. Furthermore, the mixture showed a phase separation after 5 days at 22° C.

The active content was 33.5% with a ratio of the components A to B of 1 to 1.43.

### Example 11 Not in Accordance With the Invention

A solution of 96.5 g of a fatty acid amidopropyl betaine based on refined coconut oil (containing 6.1% oleic acid acyl radicals and 1.5% linoleic acid acyl radicals based on all acyl radicals of the betaine, further composed of 54.2% water, 34.3% Cocamidopropyl Betaine (INCI), 6.8% sodium chloride, 2.6% glycerol and 2.1% fatty acids from coconut oil) and 30.0 g water was heated to 80° C. With stirring, 3.5 g of glyceryl monolaurate were added in portions within 30 min and then the mixture was stirred at 80° C. for a further 30 min. The mixture was then allowed to cool to 22° C. The product was a clear, pale yellowish liquid having a viscosity of 80 mPa·s.

The active content was 36.7% with a ratio of the components A to B of 1 to 9.46.

### Determination of the Yield Point for Examples 1-5

The yield point was determined on the basis of the guidelines for rheological tests (Thomas G. Mezger, “Das Rheologie Handbuch” [The Rheology Handbook], 2nd edition, Hannover, Vincentz Network, 2006, ISBN 3-87870-175-6).

The tests were performed on an MCR 302 Modular Compact Rheometer from Anton Paar (Graz, Austria). A PP-50 plate was used as measuring system, the temperature being controlled via a Peltier element. The temperature ramp of TEGO Betain KB 5 was measured in a cylinder geometry because of the low viscosity of the sample.

The temperature ramps were measured at a heating rate of 2° C./minute with a load of 0.2 pascals and at a frequency of 1 Hz. The frequency-dependent measurements were conducted at various temperatures between 0.1 Hz and 10 Hz, 0.2 Pa. Before the measurement, the samples were heat treated for 10 minutes.

Raw materials for the cosmetic industry are as standard stored at temperatures between 10° C. and 40° C., for which reason this defines the relevant temperature range. A sample exhibits a yield point when the storage modulus (G′) with small deflections of the sample is greater in the loss modulus (G″) of the sample. The behaviour may depend on the predefined (angular) frequency of the oscillation parameter, for which reason it is necessary to investigate whether G′>G″ at various frequencies. This testing is done at constant temperature, with a low load of 0.2 pascals (Pa) and a frequency range of from 0.1 Hz to 10 Hz. Upstream of this measurement was a measurement at constant frequency of 1 Hz (and load of 0.2 Pa) in order to test the temperature range from 0° C. to 40° C. with respect to the criterion G′>G″. If it is already the case that G′<G″ at the frequency of 1 Hz, the more complex measurement at various frequencies can be dispensed with since no yield point can be present. The ratio G′/G″ can also be represented using what is known as the loss angle. The value for the angle ranges from 0° to 90°, where G′>G″ for angles below 45°, and G′<G″ for angles greater than 45°.

The results of the temperature-dependent measurement are presented in Table 1:

| | | | | Example 1 + | | |
|---|---|---|---|---|---|---|
| | Example 5 | Example 4 | Example 1 | 10% water | Example 3 | Example 2 |
| Glyceryl | 5 | 18.6 | 8.2 | 7.5 | 9 | 12.5 |
| Monolaurate [%] | | | | | | |
| Betaine [%] | 24.7 | 27.7 | 19.3 | 17.5 | 24.9 | 21.8 |
| C18:1 + C18:2 | <2 | <2 | 18 | 18 | — | — |
| acyl radicals [%]* | | | | | | |
| Water [%] | 63.7 | 44.7 | 66.1 | 68.9 | 60.5 | 44.2 |

| Temperature | Loss angle | Loss angle | Loss angle | Loss angle | Loss angle | Loss angle |
|---|---|---|---|---|---|---|
| [° C.] | [°] | [°] | [°] | [°] | [°] | [°] |
| 0.546 | 90 | 30.9 | 33.1 | 42.2 | 70.7 | 52.3 |
| 1.67 | 90 | 23.6 | 34.8 | 39.2 | 70.7 | 41.5 |
| 2.78 | 90 | 22.9 | 28.5 | 30.8 | 71.2 | 39.4 |
| 3.86 | 90 | 29.7 | 20.7 | 23 | 71.5 | 47.5 |
| 4.92 | 90 | 30.1 | 15.5 | 14.7 | 73.6 | 58 |
| 5.93 | 90 | 31.4 | 8.52 | 7.46 | 73.7 | 66.7 |
| 6.93 | 90 | 24.8 | 6.16 | 5.48 | 74.7 | 72.9 |
| 7.93 | 90 | 32.7 | 7.49 | 7.21 | 75.6 | 77.1 |
| 8.92 | 90 | 34.9 | 9.82 | 9.97 | 76.3 | 80.3 |
| 9.9 | 90 | 38.8 | 12.9 | 15 | 76.9 | 82.5 |
| 10.9 | 90 | 42.1 | 17.9 | 23.3 | 76.4 | 84.1 |
| 11.8 | 90 | 41.7 | 24.8 | 35.5 | 75.1 | 85.3 |
| 12.8 | 90 | 30.3 | 35.4 | 48.7 | 75.2 | 86.2 |
| 13.8 | 90 | 24.3 | 47.1 | 61.7 | 76.2 | 86.9 |
| 14.8 | 90 | 20 | 58.7 | 70.6 | 77.1 | 87.4 |
| 15.8 | 90 | 19.3 | 67.6 | 76.5 | 76.2 | 87.9 |
| 16.7 | 90 | 17.4 | 74.1 | 80.4 | 72.7 | 88.2 |
| 17.7 | 90 | 17.4 | 78.4 | 82.9 | 67.6 | 88.4 |
| 18.7 | 90 | 13.3 | 82 | 84.6 | 61.7 | 88.7 |
| 19.7 | 90 | 16.9 | 83.5 | 85.8 | 54.8 | 88.9 |
| 20.6 | 90 | 16.9 | 84.9 | 86.6 | 48.7 | 89 |
| 21.6 | 90 | 17.3 | 85.9 | 87.2 | 43.9 | 89.2 |
| 22.6 | 90 | 17.4 | 86.7 | 87.6 | 40.2 | 89.3 |
| 23.6 | 90 | 17.4 | 87.3 | 87.9 | 37.5 | 89.4 |
| 24.5 | 90 | 17.2 | 87.6 | 88.2 | 35.5 | 89.4 |
| 25.5 | 90 | 17.2 | 88.1 | 88.4 | 33.8 | 89.5 |
| 26.5 | 90 | 17.2 | 88.3 | 88.6 | 32.9 | 89.6 |
| 27.5 | 90 | 16.7 | 88.6 | 88.7 | 33.2 | 89.6 |
| 28.4 | 90 | 16.6 | 88.8 | 88.8 | 33.5 | 89.7 |
| 29.4 | 90 | 16.4 | 88.9 | 88.9 | 34.1 | 89.8 |
| 30.4 | 90 | 16.6 | 89 | 88.9 | 35 | 89.8 |
| 31.4 | 90 | 16.7 | 89 | 88.8 | 36.3 | 89.9 |
| 32.3 | 90 | 13.2 | 89.2 | 88.8 | 36.1 | 89.9 |
| 33.3 | 90 | 16.4 | 89.1 | 88.5 | 37.7 | 89.9 |
| 34.3 | 90 | 16.4 | 89 | 87.7 | 38.3 | 90 |
| 35.2 | 90 | 16.2 | 88.6 | 86.9 | 39.4 | 90 |
| 36.2 | 90 | 15.8 | 88.4 | 86.2 | 40.8 | 90 |
| 37.2 | 90 | 16.3 | 88 | 85.6 | 42.6 | 90 |
| 38.2 | 90 | 16.2 | 87.7 | 85.3 | 41.6 | 90 |
| 39.1 | 90 | 16.9 | 87.3 | 85.1 | 41.4 | 90 |
| 40.1 | 90 | 15.4 | 86.9 | 85.1 | 41.7 | 90 |

*based on all acyl radicals in the component B2)

For the sake of better legibility, phase angles greater than 45° (i.e. G′<G″) are presented in normal font whereas phase angles smaller than 45° (i.e. G′>G″) are presented in italics.

It is striking that all phase angles of the sample from Example 4 are much smaller than 45°, whereas the other substances in the temperature range investigated predominantly have phase angles of greater than 45°, which means that these samples cannot have a yield point in this temperature range.

The phase angle was then measured at 10° C. at various frequencies for all samples except for the sample from Example 5 since this had a phase angle of 90° over the entire temperature range. The results are shown in Table 2:

| Frequency/ | Example 4, | Example 1, | Example 1 + 10% | Example 2, | Example 3, |
|---|---|---|---|---|---|
| Hz | 10° C. | 10° C. | water, 10° C. | 10° C. | 10° C. |
| 0.1 | 34.4 | 77.3 | 80.5 | 89 | 65.9 |
| 0.125 | 38.8 | 69.7 | 77.7 | 88.7 | 63.1 |
| 0.156 | 38.9 | 65.2 | 74.7 | 88.6 | 59.6 |
| 0.195 | 38.6 | 60.1 | 71.4 | 88.5 | 57.8 |
| 0.244 | 38.6 | 54.2 | 67.1 | 88.3 | 56.1 |
| 0.305 | 38.6 | 48.9 | 62.2 | 88.1 | 55.6 |
| 0.381 | 39.2 | 42.9 | 56.8 | 87.8 | 55.7 |
| 0.476 | 39 | 37.8 | 51 | 87.4 | 55.6 |
| 0.595 | 39.1 | 32.4 | 45.7 | 87 | 56.1 |
| 0.743 | 39.2 | 28.7 | 39.6 | 86.4 | 56.2 |
| 0.928 | 39.5 | 24.3 | 34.7 | 85.7 | 56.3 |
| 1.16 | 39.3 | 21.7 | 29.5 | 84.8 | 55.8 |
| 1.45 | 39.7 | 18.7 | 25.7 | 83.7 | 54.9 |
| 1.81 | 39.3 | 16.6 | 22 | 82.3 | 53.8 |
| 2.26 | 39.4 | 15.3 | 19.7 | 80.6 | 52 |
| 2.83 | 38.8 | 14.3 | 17 | 78.5 | 50.2 |
| 3.53 | 38.9 | 13 | 15.3 | 75.9 | 48.7 |
| 4.42 | 39 | 12.1 | 14.4 | 72.7 | 47 |
| 5.52 | 38.8 | 11.8 | 12.9 | 69.2 | 45.8 |
| 6.9 | 39.4 | 11.5 | 12.6 | 65.2 | 44.6 |
| 8.62 | 38.9 | 11.5 | 11.8 | 61.2 | 43.4 |
| 10.8 | 39.2 | 11.7 | 11.9 | 56.9 | 42.5 |

In the temperature-dependent measurement that was conducted at 1 Hz, both samples from Example 1 showed, with and without the addition of 10% by weight of water, that the substances could potentially have a yield point; however, the frequency-dependent measurement shows that the criterion no longer exists at frequencies below 1 Hz. In contrast, the sample comprising HS 60 exhibits a phase angle of 38°-39°, that is to say smaller than 45°, over all frequencies.

The same measurements were then conducted at 25° C., and the results are shown in Table 3:

| Frequency/ | Example 4, | Example 1, | Example 1 + 10% | Example 2, | Example 3, |
|---|---|---|---|---|---|
| Hz | 25° C. | 25° C. | water, 25° C. | 25° C. | 25° C. |
| 0.1 | 37.1 | 88.2 | 88.2 | 87.4 | 42.5 |
| 0.125 | 24 | 88.3 | 89.5 | 87.8 | 37 |
| 0.156 | 21.8 | 88.3 | 89.4 | 87.9 | 34.6 |
| 0.195 | 21.1 | 88.3 | 89.4 | 88.1 | 33.7 |
| 0.244 | 20.8 | 88.3 | 89.3 | 88.3 | 32.7 |
| 0.305 | 20.8 | 88.3 | 89.3 | 88.5 | 30.3 |
| 0.381 | 20.6 | 88.2 | 89.3 | 88.7 | 28 |
| 0.476 | 20.9 | 88.2 | 89.2 | 88.9 | 28.6 |
| 0.595 | 21.4 | 88.1 | 89.2 | 89.1 | 29.8 |
| 0.743 | 21.6 | 88 | 89.1 | 89.3 | 29.3 |
| 0.928 | 22.1 | 87.8 | 89.1 | 89.5 | 35.8 |
| 1.16 | 22.5 | 87.7 | 89 | 89.8 | 34.8 |
| 1.45 | 22.9 | 87.4 | 88.9 | 90 | 30.9 |
| 1.81 | 23.6 | 87.1 | 88.9 | 90 | 34.1 |
| 2.26 | 24.1 | 86.7 | 88.8 | 90 | 35 |
| 2.83 | 26.4 | 86.2 | 88.7 | 90 | 34.6 |
| 3.53 | 26 | 85.5 | 88.7 | 90 | 37 |
| 4.42 | 26.9 | 84.8 | 88.5 | 90 | 37.7 |
| 5.52 | 27.9 | 83.7 | 88.4 | 90 | 38.5 |
| 6.9 | 29 | 82.6 | 88.5 | 90 | 42.7 |
| 8.62 | 30.5 | 81.1 | 88.2 | 90 | 41.3 |
| 10.8 | 32.3 | 79.5 | 88.4 | 90 | 43.7 |

Both samples from Example 4 and Example 3 show that the phase angle is below 45°, and these samples therefore have a yield point in this range. However, it is clear from Table 1 for the sample from Example 3 that for temperatures lower than 20° C. the yield point has definitively disappeared, since the sample below this temperature has a phase angle of greater than 45° and hence G′<G″.

Lastly, the sample from Example 4 was subjected to a further frequency-dependent measurement at 40° C. in order to check whether at this temperature G′>G″ or whether it has a phase angle of less than 45°. As can be seen in Table 4, the phase angle at all three temperatures investigated for the sample from Example 4 is less than 45°, and therefore the sample from Example 4 has a yield point in the temperature range between 10° C. and 40° C.

TABLE 4

| Frequency/ | Example 4, | Example 4, | Example 4, |
|---|---|---|---|
| Hz | 10° C. | 25° C. | 40° C. |
| 0.1 | 34.4 | 37.1 | 28.5 |
| 0.125 | 38.8 | 24 | 26.4 |
| 0.156 | 38.9 | 21.8 | 25 |
| 0.195 | 38.6 | 21.1 | 24.3 |
| 0.244 | 38.6 | 20.8 | 24 |
| 0.305 | 38.6 | 20.8 | 23.7 |
| 0.381 | 39.2 | 20.6 | 23.7 |
| 0.476 | 39 | 20.9 | 24 |
| 0.595 | 39.1 | 21.4 | 24.1 |
| 0.743 | 39.2 | 21.6 | 24.4 |
| 0.928 | 39.5 | 22.1 | 24.6 |
| 1.16 | 39.3 | 22.5 | 25.5 |
| 1.45 | 39.7 | 22.9 | 26.3 |
| 1.81 | 39.3 | 23.6 | 27 |
| 2.26 | 39.4 | 24.1 | 27.9 |
| 2.83 | 38.8 | 26.4 | 28.7 |
| 3.53 | 38.9 | 26 | 29.7 |
| 4.42 | 39 | 26.9 | 30.8 |
| 5.52 | 38.8 | 27.9 | 32.1 |
| 6.9 | 39.4 | 29 | 33.8 |
| 8.62 | 38.9 | 30.5 | 35.6 |
| 10.8 | 39.2 | 32.3 | 37.9 |

### Thickening Performance in Simple Cleansing Formulations

The formulations specified below were produced by mixing the individual components with water while stirring at 22° C., whereby water was added in amount that was needed to achieve 95% by weight based on the final formulation composition. After 1 hour of stirring, a composition according to Examples 1 to 5, 8, 9 and 11 was added in each case as a thickener, the pH was adjusted to 5.2 with citric acid and the formulation was filled up to 100% by weight with water. The viscosity was measured, after homogenization and at least 24 hours of resting time at 22° C., with a Brookfield viscometer using spindle 62 at 30 revolutions per minute and at a temperature of 22° C. Examples 6, 7 and 10 were excluded from the tests, because of phase separation issues.

All percentages given below are weight percentages.

TABLE 5
Thickening performance of Examples 1 to 5, 8, 9 and 11 in cleansing formulation A comprising
32% of a 28% aq. sodium lauryl ether sulfate solution (Texapon NSO-IS; SLES 28%),
8% of a 38% aq. cocamidopropyl betaine solution (TEGO Betain F 50) and 0.7% sodium
chloride, and in cleansing formulation B comprising 17.5% of a 32% aq. sodium cocoamphoacetate
solution (REWOTERIC AM C), 8.8% of a 50% aq. lauryl glucoside solution (Plantacare
1200 UP), 4.4% of a 50% aq. coco glucoside solution (Plantacare 818 UP) and 14.4%
of a 25% aq. cocoyl glutamate solution (Perlastan SC 25 NKW).

| | Example 1 | Example 2 | Example 3 | Example 4 | Example 5 |
|---|---|---|---|---|---|
| Active content of | 33.9 | 43.3 | 39.5 | 55.5 | 36.4 |
| example / % | | | | | |
| Viscosity of | 3500 | 950 | 600 | 3400 | 80 |
| example / mPa · s | | | | | |
| Appearance of | clear, | clear, | clear, | turbid, | clear, |
| example | homogeneous, | homogeneous, | homogeneous, | liquid | homogeneous, |
| | liquid | liquid | liquid | | liquid |
| Use amount of | 3.3 | 2.1 | 2.7 | 2.0 | 3.0 |
| example in base | | | | | |
| formulation A / % | | | | | |
| Active content of | 1.1 | 0.9 | 1.1 | 1.1 | 1.1 |
| example in base | | | | | |
| formulation A / % | | | | | |
| Viscosity of base | 3900 | 2735 | 6000 | 4100 | 1120 |
| formulation A / | | | | | |
| mPa · s | | | | | |
| Use amount of | 3.4 | — | — | 2.0 | 3.0 |
| example in base | | | | | |
| formulation B / % | | | | | |
| Active content of | 1.1 | — | — | 1.1 | 1.1 |
| example in base | | | | | |
| formulation B / % | | | | | |
| Viscosity of base | 4000 | — | — | 1800 | 500 |
| formulation B / | | | | | |
| mPa · s | | | | | |

| | Example | Example | Example | Example | Example | Example |
|---|---|---|---|---|---|---|
| | 6 | 7 | 8 | 9 | 10 | 11 |
| Active content of | 55.4 | 34.5 | 35.9 | 34.5 | 33.5 | 36.7 |
| example / % | | | | | | |
| Viscosity of | 6700 | 2200 | 90 | 3100 | 1750 | 80 |
| example / mPa · s | | | | | | |
| Appearance of | turbid | turbid | clear, | clear, | turbid | clear, |
| example | liquid, | liquid, | homogeneous, | homogeneous, | liquid, | homogeneous, |
| | phase | phase | liquid | liquid | phase | liquid |
| | separation | separation | | | separation | |
| Use amount of | — | — | 3.1 | 3.2 | — | 3.0 |
| example in base | | | | | | |
| formulation A / % | | | | | | |
| Active content of | — | — | 1.1 | 1.1 | — | 1.1 |
| example in base | | | | | | |
| formulation A / % | | | | | | |
| Viscosity of base | — | — | 1340 | 2950 | — | 885 |
| formulation A / | | | | | | |
| mPa · s | | | | | | |
| Use amount of | — | — | 3.1 | 3.2 | — | 3.0 |
| example in base | | | | | | |
| formulation B / % | | | | | | |
| Active content of | — | — | 1.1 | 1.1 | — | 1.1 |
| example in base | | | | | | |
| formulation B / % | | | | | | |
| Viscosity of base | — | — | 640 | 2635 | — | 380 |
| formulation B / | | | | | | |
| mPa · s | | | | | | |

The amount of the compositions according to the examples 1 to 5, 8, 9 and 11 indicated in the table was added to both cleansing formulations. The respective amount of water added is given by the difference between 100% and the sum of all feedstocks.

### Thickening Performance in Relatively Complex Guideline Formulations

The viscosity measurements were conducted with a Brookfield viscometer (Brookfield RVDV-I Prime) under the following conditions:
Temperature: 23° C.
Spindle: LV 2
Rotation speed: 30 RPM

### Guideline Formulation 1: Anti-Dandruff Shampoo

| | | GF1A | GF1B | GF1C | GF1D | GF1E | GF1F | GF1G | GF1H |
|---|---|---|---|---|---|---|---|---|---|
| A | Tegin G 1100 | 3 | 3 | 3 | 3 | 3 | 3 | 3 | 3 |
| | pellets / % | | | | | | | | |
| | SLES 28% / % | 40 | 40 | 40 | 40 | 40 | 40 | 40 | 40 |
| B | perfume pure | 0.3 | 0.3 | 0.3 | 0.3 | 0.3 | 0.3 | 0.3 | 0.3 |
| | sensation / % | | | | | | | | |
| | zinc pyrithione | 2 | 2 | 2 | 2 | 2 | 2 | 2 | 2 |
| | NF / % | | | | | | | | |
| | ABIL B 88183 / % | 0.5 | 0.5 | 0.5 | 0.5 | 0.5 | 0.5 | 0.5 | 0.5 |
| C | water, demin. / % | 37.5 | 38.8 | 38.4 | 40 | 37.9 | 37.9 | 37.6 | 38.0 |
| | Tego Carbomer | 0.2 | 0.2 | 0.2 | 0.2 | 0.2 | 0.2 | 0.2 | 0.2 |
| | 141 / % | | | | | | | | |
| | NaOH 25% / % | 0.3 | 0.3 | 0.3 | 0.3 | 0.3 | 0.3 | 0.3 | 0.3 |
| D | TEGO Betain F | 10 | 10 | 10 | 10 | 10 | 10 | 10 | 10 |
| | 50 / % | | | | | | | | |
| | Example 1 / % | 6.2 | | | | | | | |
| | Example 2 / % | | 4.9 | | | | | | |
| | Example 3 / % | | | 5.3 | | | | | |
| | Example 4 / % | | | | 3.7 | | | | |
| | Example 5 / % | | | | | 5.8 | | | |
| | Example 8 / % | | | | | | 5.8 | | |
| | Example 9 / % | | | | | | | 6.1 | |
| | Example 11 / % | | | | | | | | 5.7 |
| | pH | 5.5 | 5.5 | 5.5 | 5.5 | 5.5 | 5.5 | 5.5 | 5.5 |
| | Viscosity /mPa · s | 8779 | 9427 | 8917 | 6827 | 5527 | 5123 | 6331 | 3272 |
| | (spindle 2 / 30 rpm) | | | | | | | | |
| | Appearance | white | white | white | white | white | white | white | white |
| | Batch size / g | 150 | 150 | 150 | 150 | 150 | 150 | 150 | 150 |
| | Active content of | 2.1 | 2.1 | 2.1 | 2.1 | 2.1 | 2.1 | 2.1 | 2.1 |
| | example / % | | | | | | | | |

Preparation: SLES and TEGIN G 1100 are heated to 65° C. and then cooled down slowly.

Constituents B were added successively to phase A. TEGO Carbomer is incorporated in water with stirring and neutralized with NaOH. Phase C is then added to phase AB. Lastly, the remaining raw materials are added in the indicated sequence and the pH is adjusted.

Comparison of the formulation viscosities based on examples.

| | Viscosity mPa · s | |
|---|---|---|
| | (SP2/30 rpm) | |
| Example 1 | 8779 | According to the invention |
| Example 2 | 9427 | According to the invention |
| Example 3 | 8917 | According to the invention |
| Example 4 | 6827 | Not in accordance with the invention |
| Example 5 | 5527 | Not in accordance with the invention |
| Example 8 | 5123 | Not in accordance with the invention |
| Example 9 | 6331 | According to the invention |
| Example 11 | 3272 | Not in accordance with the invention |

### Guideline Formulation 2: Pearlescent Liquid Soap

| | GF2A | GF2B | GF2C | GF2D | GF2F | GF2G | GF2H | GF2I |
|---|---|---|---|---|---|---|---|---|
| SLES 28% / % | 40.0 | 40.0 | 40.0 | 40.0 | 40.0 | 40.0 | 40.0 | 40.0 |
| TEGOSOFT LSE | 3.0 | 3.0 | 3.0 | 3.0 | 3.0 | 3.0 | 3.0 | 3.0 |
| 65 K Soft / % | | | | | | | | |
| Perfume / % | 0.2 | 0.2 | 0.2 | 0.2 | 0.2 | 0.2 | 0.2 | 0.2 |
| water, demin. / % | 41.15 | 43.5 | 44.45 | 43.15 | 43.05 | 42.85 | 43.15 | 42.75 |
| TEGO Betain F | 6.4 | 6.4 | 6.4 | 6.4 | 6.4 | 6.4 | 6.4 | 6.4 |
| 50 / % | | | | | | | | |
| Tego Pearl N | 3.0 | 3.0 | 3.0 | 3.0 | 3.0 | 3.0 | 3.0 | 3.0 |
| 100 / % | | | | | | | | |
| Preservative / % | 0.45 | 0.45 | 0.45 | 0.45 | 0.45 | 0.45 | 0.45 | 0.45 |
| Example 1 + 10% | 5.8 | | | | | | | |
| H₂O | | | | | | | | |
| Example 1 | | | | | | | | 4.2 |
| Example 3 / % | | 3.5 | | | | | | |
| Example 4 / % | | | 2.5 | | | | | |
| Example 5 / % | | | | 3.8 | | | | |
| Example 8 / % | | | | | 3.9 | | | |
| Example 9 / % | | | | | | 4.1 | | |
| Example 11 / % | | | | | | | 3.8 | |
| pH | 5.5 | 5.5 | 5.5 | 5.5 | 5.5 | 5.5 | 5.5 | 5.5 |
| Viscosity / mPa · s | 7723 | 1429 | 3296 | 1003 | 1238 | 2761 | 773 | 3683 |
| (SP2 / 30 rpm) | | | | | | | | |
| Appearance | pearle | pearle | pearle | pearle | pearle | pearle | pearle | pearle |
| | scent | scent | scent | scent | scent | scent | scent | scent |
| Batch size / g | 150 | 150 | 150 | 150 | 150 | 150 | 150 | 150 |
| Active content of | 1.8 | 1.4 | 1.45 | 1.4 | 1.4 | 1. | 1.4 | 1.4 |
| example / % | | | | | | | | |

Preparation: The formulation constituents are mixed in the indicated sequence with stirring. The pH is then adjusted with citric acid.

### Comparison of the Viscosities

| | Viscosity mPa · s | |
|---|---|---|
| | (spindle 2/30 rpm) | |
| Example 1 + | 7723 (1.8%) | According to the invention |
| 10% water | | |
| Example 1 | 3683 (1.4%) | According to the invention |
| Example 3 | 1429 (1.4%) | According to the invention |
| Example 4 | 3296 (1.4%) | Not in accordance with the invention |
| Example 5 | 1003 (1.4%) | Not in accordance with the invention |
| Example 8 | 1238 (1.4%) | Not in accordance with the invention |
| Example 9 | 2761 (1.4%) | According to the invention |
| Example 11 | 773 (1.4%) | Not in accordance with the invention |

1. A composition, comprising:
A) at least one glycerol fatty acid ester and
B) at least one betaine component selected from the group consisting of
B1) fatty acid amidoalkyl betaine of the general formula I) 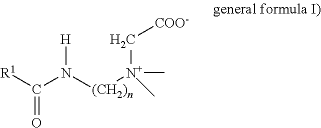
wherein
n=1 to 10, and
R¹CO=a mixture of acyl radicals having 6 to 30 carbon atoms,
wherein the mixture of acyl radicals has a total content of oleic acid acyl radicals and linoleic acid acyl radicals, based on all acyl radicals of the mixture, of 5% by weight to 50% by weight, and
B2) alkyl betaine of the general formula II) 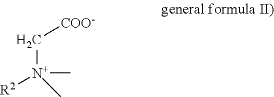
wherein
R²=saturated or unsaturated, optionally hydroxy-substituted alkyl radical having 6 to 22 carbon atoms, or mixtures thereof,
wherein a weight ratio of all glycerol fatty acid esters present in the composition to all fatty acid amidoalkyl betaines and alkyl betaines present in the composition is from 1.0:1.5 to 1.0:8.0.

2. The composition according to claim 1, which comprises 3.0% by weight to 20% by weight of glycerol fatty acid esters, wherein the weight percentages are based on the composition.

3. The composition according to claim 1, which comprises in total 10% by weight to 35% by weight of fatty acid amidoalkyl betaines and alkyl betaines, where the weight percentages are based on the composition.

4. The composition according to claim 1, wherein the at least one glycerol fatty acid ester, based on all acyl radicals present in the at least one glycerol fatty acid ester, includes at least 90% by weight acyl radicals having 8 to 18 carbon atoms.

5. The composition according to claim 1, wherein the mixture of acyl radicals in the fatty acid amidoalkyl betaine of the general formula I) has a content of acyl radicals having 8 to 18 carbon atoms of at least 90% by weight, wherein the weight percentages are based on all acyl radicals in the mixture.

6. The composition according to claim 1, wherein the at least one betaine component is the alkyl betaine of the general formula II), and wherein R² in the alkyl betaine of the general formula II) includes in total at least 50% by weight lauryl, myristyl, and cetyl radicals, wherein the weight percentages are based on all alkyl radicals in the alkyl betaines present in the composition.

7. The composition according to claim 1, further comprising 33% by weight to 83% by weight of water, wherein the weight percentages are based on the composition.

8. The composition according to claim 1, further comprising in total 0.1% by weight to 10% by weight of sodium chloride and/or potassium chloride, wherein the weight percentages are based on the composition.

9. The composition according to claim 1, further comprising 0.1% by weight to 25% by weight of glycerol, wherein the weight percentages are based on the composition.

10. The composition according to claim 1, further comprising in total 0.1% by weight to 3% by weight of fatty acids selected from fatty acids having 8 to 18 carbon atoms, wherein the weight percentages are based on the composition.

11. The composition according to claim 1, which does not have a yield point between 10 and 40° C.

12. A cosmetic, pharmaceutical, or dermatological formulation, comprising:
at least one composition according to claim 1.

13. A method for thickening of a formulation, the method comprising: mixing the formulation with Use of at least one composition according to claim 1.

14. The composition according to claim 1, wherein the at least one betaine component is the fatty acid amidoalkyl betaine of the general formula I), and wherein in the fatty acid amidoalkyl betaine of the general formula I), n=3.

15. The composition according to claim 1, wherein the mixture of acyl radicals has a total content of oleic acid acyl radicals and linoleic acid acyl radicals, based on all acyl radicals of the mixture, of 13% by weight to 20% by weight.

16. The composition according to claim 1, which comprises 6.5% by weight to 10% by weight of glycerol fatty acid esters, wherein the weight percentages are based on the composition.

17. The composition according to claim 1, which comprises 14% by weight to 27% by weight of fatty acid amidoalkyl betaines and alkyl betaines, wherein the weight percentages are based on the composition.

18. The composition according to claim 1, wherein the at least one betaine component is the alkyl betaine of the general formula II), and
wherein R² in the alkyl betaine of the general formula II) includes in total at least 50% by weight lauryl radicals, wherein the weight percentages are based on all alkyl radicals in the alkyl betaines present in the composition.

19. The composition according to claim 1, further comprising 48% by weight to 78% by weight of water, wherein the weight percentages are based on the composition.

20. The method according to claim 13, wherein the formulation is an aqueous formulation.
